(19) United States
(12) Patent Application Publication
(10) Pub. No.: US 2018/0055915 A1
(43) Pub. Date: Mar. 1, 2018

(54) COMPOSITIONS AND METHODS FOR TREATING PROSTATE CANCER

(71) Applicant: UNIVERSITY OF ROCHESTER, ROCHESTER, NY (US)

(72) Inventors: Chawnshang Chang, Pittsford, NY (US); Shu-Yuan Yeh, Rochester, NY (US)

(21) Appl. No.: 15/456,169

(22) Filed: Mar. 10, 2017

Related U.S. Application Data
(60) Provisional application No. 62/307,376, filed on Mar. 11, 2016.

(51) Int. Cl. A61K 38/46; A61K 39/00; C12Q 1/68

(52) U.S. Cl. CPC A61K 38/465; C12Q 2600/158; C12Q 1/6886; A61K 39/0011

(57) ABSTRACT

The invention provides novel therapeutically methods and pharmaceutical compositions for treating prostate cancer with increased survival rate and improved treatment outcome. Methods and compositions of the invention can be used to prevent, delay, reduce and/or drug resistance and to increase, restore and/or prolong the effective treatment of prostate cancer with anti-androgen compounds.

### PRIORITY CLAIMS AND RELATED APPLICATIONS

This application claims the benefit of priority from U.S. Provisional Application No. 62/307,376, filed Mar. 11, 2016, the entire content of which is incorporated herein by reference for all purposes.

### GOVERNMENT RIGHTS

This invention was made with government support under Grant No. CA156700 and CA127300 awarded by the NIH. The Government has certain rights in the invention.

### TECHNICAL FIELD OF THE INVENTION

The invention generally relates to novel therapeutic methods and pharmaceutical compositions for treating cancer. More particularly, the invention relates to a novel approach to addressing drug resistance in prostate cancer treatment.

### BACKGROUND OF THE INVENTION

Prostate cancer is the most common form of cancer and the second leading cause of cancer death among men in the United States. (Centers for Disease Control and Prevention. http://www.cdc.gov/cancer/dcpc/data/men.htm.) Approximately one in seven men will be diagnosed with prostate cancer during his lifetime, and about one in 38 men will die of the disease with an estimated 307,000 deaths worldwide in 2012. (Merseburger, et al. 2015 Ther Adv Urol 7, 9-21.)

The current standard treatment for advanced prostate cancer is androgen deprivation therapy (ADT), which may suppress prostate cancer progression by reducing androgen synthesis or by preventing androgen from binding to the androgen receptor. (Chang, et al. 1988 Science 240, 324-326; Heinlein, et al. 2004 Endocr Rev 25, 276-308; Chang, et al. 2014 Oncogene 33, 3225-3234; Niu, et al. 2010 Oncogene 29, 3593-3604.)

A newly developed anti-androgen compound, Enzalutamide (Enz), was showed to suppress castration resistant prostate cancer (CRPC) and could further extend patients overall survival by an average of 4.8 months. (Scher, et al., 2012 N Engl J Med 367, 1187-1197.) Nonetheless, patients eventually developed Enz resistance (EnzR) thereafter. (Antonarakis, et al. 2014 N Engl J Med 371, 1028-1038.)

Currently, there is not an effective way to delay, reduce or reverse such drug resistance, which has seriously limited the overall effectiveness and treatment outcome of anti-androgen therapy. There remains an urgent need for novel and improved approaches that effectively address these issues.

### SUMMARY OF THE INVENTION

The invention is based in part on the unexpected discovery that Cisplatin (Cis) can be used to prevent, delay, reduce and/or EnzR resistance and to increase, restore and/or prolong the effective treatment of prostate cancer with anti-androgen compounds, such as Enz. As disclosed herein, Cis delays the onset and development and/or reduces the magnitude of EnzR and suppresses EnzR tumors by targeting androgen receptor (AR) mutants and splicing variants (e.g., androgen receptor splice variant-7, or ARv7 or AR-V7).

The present invention may fundamentally alter the treatment protocol for prostate cancer patients. Therapeutic methods and pharmaceutical compositions of the invention may be used to treat prostate cancer, or a related disease or condition, in ways that minimize the risk of drug resistance while maximizing treatment outcome.

In one aspect, the invention generally relates to a pharmaceutical composition, which include: a first compound of Formula (I)

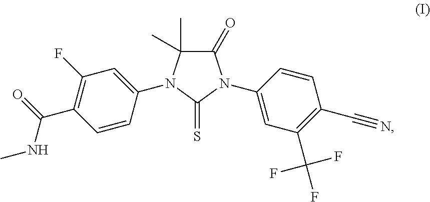 [SMILES: CNC(=O)c1ccc(N2C(=S)N(c3ccc(C#N)c(C(F)(F)F)c3)C(=O)C2(C)C)cc1F]

or a pharmaceutically acceptable salt, ester or pro-drug thereof, and a second compound of Formula (II)

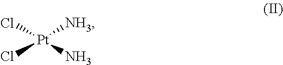

or a pro-drug thereof, wherein each of the first and second compounds is present in an amount such that the pharmaceutical composition is effective in the treatment of prostate cancer, or a related disease or condition thereof, in a mammal, including a human, and a pharmaceutically acceptable carrier.

In certain embodiments of the pharmaceutical composition, each of the first and second compounds is present in an amount such that the pharmaceutical composition is effective in preventing the onset of or delaying the development of drug resistance.

In certain embodiments, each of the first and second compounds is present in an amount such that the pharmaceutical composition is effective in reducing or reversing drug resistance.

In another aspect, the invention generally relates to a unit dosage form comprising the pharmaceutical composition disclosed herein.

In yet another aspect, the invention generally relates to a method for treating prostate cancer, or a related disease or condition thereof. The method includes administering to a subject in need thereof the pharmaceutical composition disclosed herein.

In yet another aspect, the invention generally relates to a method for treating prostate cancer, or a related disease or condition thereof. The method includes administering to a subject in need thereof a first compound of Formula (I):

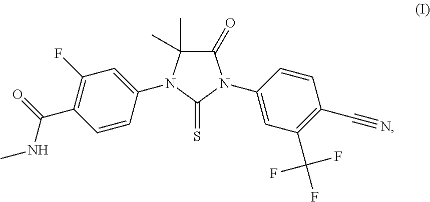 [SMILES: CNC(=O)c1ccc(N2C(=S)N(c3ccc(C#N)c(C(F)(F)F)c3)C(=O)C2(C)C)cc1F]

or a pharmaceutically acceptable salt, ester or pro-drug thereof, and a second compound of Formula (II)

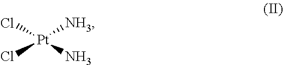

or a pro-drug thereof, in amounts effective in the treatment of prostate cancer, or a related disease or condition thereof, in a mammal, including a human.

In yet another aspect, the invention generally relates to a method for treating prostate cancer, or a related disease or condition thereof. The method includes: administering to a subject in need thereof a first compound of Formula (I)

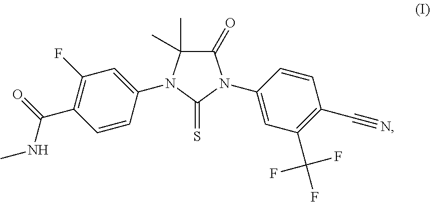 [SMILES: CNC(=O)c1ccc(N2C(=S)N(c3ccc(C#N)c(C(F)(F)F)c3)C(=O)C2(C)C)cc1F]

or a pharmaceutically acceptable salt, ester or pro-drug thereof, in an amount effective in the treatment of prostate cancer, or a related disease or condition thereof, in a mammal, including a human; monitoring the subject to detect a development of drug resistance to the first compound; upon the subject being detected of the development of drug resistance to the first compound, administering to the subject a second compound of Formula (II)

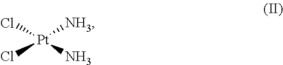

or a pro-drug thereof, in an amount effect to reduce or eliminate drug resistance to the first compound; and monitoring the subject to detect a level of drug resistance to the first compound.

In yet another aspect, the invention generally relates to a method for treating a drug resistance in connection with a cancer treatment. The method includes administering to a subject in need thereof a compound of Formula (II)

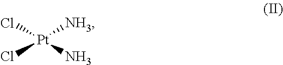

or a pro-drug thereof, in an amount effective to prevent, delay, reduce or reverse resistance to a treatment of prostate cancer, or a related disease or condition thereof, in a mammal, including a human.

### BRIEF DESCRIPTION OF THE DRAWINGS

FIG. 1. Cisplatin degrades AR wild type, mutant and ARv7 expression in CRPCs. AR/ARv7 expression in (A) EnzS1 C4-2 (B) EnzR1 C4-2 (C) EnzS3 C4-2B (D) Enz R3 C4-2B (E) EnzR2 CWR22Rv1 and (F) Enz R2 CWR22Rv1 treated with carbonplatin (G) PC3-AR F876L.

FIG. 2. Cisplatin postpones/delays enzalutamide resistance developing. Cisplatin restores enzalutamide sensitivity in EnzR prostate cancer cells. One EnzR PCa cell line was established in C4-2 cells (named as EnzR1C4-2) and another cell line was set up for EnzR3 C4-2B. Survival rate of EnzR1 C4-2 cells under Enz (A), Cis/Cis+Enz (B) and IC50 (C); Survival rate of EnzR2 CWR22Rv1 under Enz (D), and IC50 (E); Survival rate of EnzR2 C4-2B under Enz (F), Cis/Cis+Enz (G) and IC50 (H); cPARP level in EnzR1 C4-2(I), EnzR3 C4-2B (J).

FIG. 3. Enz C4-2 cells were treated with Enz or Enz+Cis for 2 months (A-C). Enz sensitivity was analyzed by measuring proliferation rate using MTT assay. (A) EnzS1 C4-2 (B) EnzS1 C4-2 w/Enz; (C) EnzS1 C4-2 w/Enz+Cis (0.2 mg/mL); (D) mRNA level and protein level w/Enz+Cis (0.2 ug/mL) treatment for 1 month (DMSO, Enz, Enz+Cis 0.2 ug/mL).

FIG. 4. Cisplatin degrades ARv7 expression through increasing AR ubiquitination in EnzR C4-2 cells. (A) EnzR1 C4-2 cells were treated with 1 ug/mL cisplatin for 24 hours (B) EnzR1 C4-2 cells were treated with 1 ug/mL cisplatin for 6 hours and treated with MG132 for another 6 hrs. (C) EnzR1 C4-2 cells were treated with/without 1 ug/mL cisplatin for 6 hrs and treated with cycloheximide for another 6 hrs. (D) pGFP-ubiquitin/pAR transfected HEK293T cells were treated with 1 ug/mL cisplatin for 6 hours and treated with MG132 for another 4 hrs. Protein was extracted and analyzed by using western blot. (E-F) EnzR1 C4-2 cells were treated with 1 ug/mL cisplatin for 24 hours. ARv7 (E) and MALAT1 (F) mRNA was extracted and analyzed by using real-time PCR. (G) RP11-47311.9 mRNA level.

FIG. 5. Cisplatin decreases AR mRNA via MALAT1/SF2-independent pathway. EnzR C4-2 cells were treated with 1 ug/mL cisplatin for 24 hr. (A) AR mRNA expression after treating cisplatin by using real-time PCR. (B) AR promoter activity decreased after cisplatin treatment.

### DEFINITIONS

Unless defined otherwise, all technical and scientific terms used herein have the same meaning as commonly understood by one of ordinary skill in the art to which this invention belongs. General principles of organic chemistry, as well as specific functional moieties and reactivity, are described in “Organic Chemistry”, Thomas Sorrell, University Science Books, Sausalito: 2006.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic methods well known in the art, and subsequent recovery of the pure enantiomers.

Cisplatin (a.k.a., cisplatinum, platamin, neoplatin, cismaplat or cis-diamminedichloroplatinum(II), Platinol, CAS No. 15663-27-1) is a chemotherapy drug. It was the first member of a class of platinum-containing anti-cancer drugs. It binds to and causes crosslinking of DNA, which ultimately triggers apoptosis (programmed cell death). The IUPAC name of the compound is (SP-4-2)-diamminedichloroplatinum(II).

Enzalutamide (a.k.a., MDV3100 and Xtandi, CAS No. 915087-33-1) is a synthetic, non-steroidal pure antiandrogen that was developed for the treatment of metastatic castration-resistant prostate cancer. The IUPAC name of the compound is 4-(3-(4-Cyano-3-(trifluoromethyl)phenyl)-5,5-dimethyl-4-oxo-2-thioxoimidazolidin-1-yl)-2-fluoro-N-methylbenzamide.

As used herein, the term “effective amount” of an active agent refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the patient.

As used herein, the terms “treatment” or “treating” a disease or disorder refers to a method of reducing, delaying or ameliorating such a condition before or after it has occurred. Treatment may be directed at one or more effects or symptoms of a disease and/or the underlying pathology. The treatment can be any reduction and can be, but is not limited to, the complete ablation of the disease or the symptoms of the disease. As compared with an equivalent untreated control, such reduction or degree of prevention is at least 5%, 10%, 20%, 40%, 50%, 60%, 80%, 90%, 95%, or 100% as measured by any standard technique.

As used herein, the terms “prevent”, “preventing”, or “prevention” refer to a method for precluding, delaying, averting, or stopping the onset, incidence, severity, or recurrence of a disease or condition. For example, a method is considered to be a prevention if there is a reduction or delay in onset, incidence, severity, or recurrence of a disease or condition or one or more symptoms thereof in a subject susceptible to the disease or condition as compared to a subject not receiving the method. The disclosed method is also considered to be a prevention if there is a reduction or delay in onset, incidence, severity, or recurrence of osteoporosis or one or more symptoms of a disease or condition in a subject susceptible to the disease or condition after receiving the method as compared to the subject's progression prior to receiving treatment. Thus, the reduction or delay in onset, incidence, severity, or recurrence of osteoporosis can be about a 5, 10, 20, 30, 40, 50, 60, 70, 80, 90, 100%, or any amount of reduction in between.

As used herein, the term “pharmaceutically acceptable” excipient, carrier, or diluent refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

As used herein, the terms “isolated” or “purified” refer to a material that is substantially or essentially free from components that normally accompany it in its native state. Purity and homogeneity are typically determined using analytical chemistry techniques such as polyacrylamide gel electrophoresis or high performance liquid chromatography.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

As used herein, the term “low dosage” refers to at least 5% less (e.g., at least 10%, 20%, 50%, 80%, 90%, or even 95%) than the lowest standard recommended dosage of a particular compound formulated for a given route of administration for treatment of any human disease or condition. For example, a low dosage of an agent that is formulated for administration by inhalation will differ from a low dosage of the same agent formulated for oral administration.

As used herein, the term “high dosage” is meant at least 5% (e.g., at least 10%, 20%, 50%, 100%, 200%, or even 300%) more than the highest standard recommended dosage of a particular compound for treatment of any human disease or condition.

As used herein, the term “prodrug” (or “pro-drug”) refers to a pharmacological derivative of a parent drug molecule that requires biotransformation, either spontaneous or enzymatic, within the organism to release the active drug. Such prodrugs are pharmaceutically active in vivo, when they undergo solvolysis under physiological conditions or undergo enzymatic degradation. Prodrug compounds herein may be called single, double, triple, etc., depending on the number of biotransformation steps required to release the active drug within the organism, and the number of functionalities present in a precursor-type form.

Prodrug forms often offer advantages of solubility, tissue compatibility, or delayed release in the mammalian organism. (See, Bundgard, Design of Prodrugs, pp. 7-9, 21-24, Elsevier, Amsterdam 1985 and Silverman, The Organic Chemistry of Drug Design and Drug Action, pp. 352-401, Academic Press, San Diego, Calif., 1992). Prodrugs commonly known in the art include well-known acid derivatives, such as, for example, esters prepared by reaction of the parent acids with a suitable alcohol, amides prepared by reaction of the parent acid compound with an amine, basic groups reacted to form an acylated base derivative, etc. Of course, other prodrug derivatives may be combined with other features disclosed herein to enhance bioavailability. As such, those of skill in the art will appreciate that certain of the presently disclosed compounds having free amino, arnido, hydroxy or carboxylic groups can be converted into prodrugs. Prodrugs include compounds having an amino acid residue, or a polypeptide chain of two or more (e.g., two, three or four) amino acid residues which are covalently joined through peptide bonds to free amino, hydroxy or carboxylic acid groups of the presently disclosed compounds. The amino acid residues include the 20 naturally occurring amino acids commonly designated by three letter symbols and also include 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Prodrugs also include compounds having a carbonate, carbamate, amide or alkyl ester moiety covalently bonded to any of the above substituents disclosed herein.

Isotopically-labeled compounds are also within the scope of the present disclosure. As used herein, an “isotopically-labeled compound” refers to a presently disclosed compound including pharmaceutical salts and prodrugs thereof, each as described herein, in which one or more atoms are replaced by an atom having an atomic mass or mass number different from the atomic mass or mass number usually found in nature. Examples of isotopes that can be incorporated into compounds presently disclosed include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

By isotopically-labeling the presently disclosed compounds, the compounds may be useful in drug and/or substrate tissue distribution assays. Tritiated (³H) and carbon-14 (¹⁴C) labeled compounds are particularly preferred for their ease of preparation and detectability. Further, substitution with heavier isotopes such as deuterium (²H) can afford certain therapeutic advantages resulting from greater metabolic stability, for example increased in vivo half-life or reduced dosage requirements and, hence, may be preferred in some circumstances. Isotopically labeled compounds presently disclosed, including pharmaceutical salts, esters, and prodrugs thereof, can be prepared by any means known in the art.

Further, substitution of normally abundant hydrogen (¹H) with heavier isotopes such as deuterium can afford certain therapeutic advantages, e.g., resulting from improved absorption, distribution, metabolism and/or excretion (ADME) properties, creating drugs with improved efficacy, safety, and/or tolerability. Benefits may also be obtained from replacement of normally abundant ¹²C with ¹³C. (See, WO 2007/005643, WO 2007/005644, WO 2007/016361, and WO 2007/016431.)

Stereoisomers (e.g., cis and trans isomers) and all optical isomers of a presently disclosed compound (e.g., R and S enantiomers), as well as racemic, diastereomeric and other mixtures of such isomers are within the scope of the present disclosure.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

Solvates and polymorphs of the compounds of the invention are also contemplated herein. Solvates of the compounds of the present invention include, for example, hydrates.

Any appropriate route of administration can be employed, for example, parenteral, intravenous, subcutaneous, intramuscular, intraventricular, intracorporeal, intraperitoneal, rectal, or oral administration. Most suitable means of administration for a particular patient will depend on the nature and severity of the disease or condition being treated or the nature of the therapy being used and on the nature of the active compound.

Solid dosage forms for oral administration include capsules, tablets, pills, powders, and granules. In such solid dosage forms, the compounds described herein or derivatives thereof are admixed with at least one inert customary excipient (or carrier) such as sodium citrate or dicalcium phosphate or (i) fillers or extenders, as for example, starches, lactose, sucrose, glucose, mannitol, and silicic acid, (ii) binders, as for example, carboxymethylcellulose, alignates, gelatin, polyvinylpyrrolidone, sucrose, and acacia, (iii) humectants, as for example, glycerol, (iv) disintegrating agents, as for example, agar-agar, calcium carbonate, potato or tapioca starch, alginic acid, certain complex silicates, and sodium carbonate, (v) solution retarders, as for example, paraffin, (vi) absorption accelerators, as for example, quaternary ammonium compounds, (vii) wetting agents, as for example, cetyl alcohol, and glycerol monostearate, (viii) adsorbents, as for example, kaolin and bentonite, and (ix) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents. Solid compositions of a similar type may also be employed as fillers in soft and hard-filled gelatin capsules using such excipients as lactose or milk sugar as well as high molecular weight polyethyleneglycols, and the like. Solid dosage forms such as tablets, dragees, capsules, pills, and granules can be prepared with coatings and shells, such as enteric coatings and others known in the art.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, such as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like. Besides such inert diluents, the composition can also include additional agents, such as wetting, emulsifying, suspending, sweetening, flavoring, or perfuming agents.

Materials, compositions, and components disclosed herein can be used for, can be used in conjunction with, can be used in preparation for, or are products of the disclosed methods and compositions. It is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

### DETAILED DESCRIPTION OF THE INVENTION

The invention provides a unique approach to treatment of prostate cancer. The novel therapeutic methods and compositions provided herein can benefit prostate cancer patients in terms of increased survival rate and improved treatment outcome. Methods and compositions of the invention can be used to prevent, delay, reduce and/or reverse drug resistance and to increase, restore and/or prolong the effective treatment of prostate cancer with anti-androgen compounds.

Enzalutamide, an anti-androgen agent, has been successfully used as the last line therapy to extend lives of castration resistant prostate cancer (CRPC) patients. After prostate cancer patients develop CRPC, the current standard therapy to further suppress CRPC involves either docetaxel (Doc)-chemotherapy or ADT with either using Enz to prevent androgens binding to AR or using abiraterone (ABI) to further suppress the androgen synthesis in renal. Unfortunately, many patients may still develop Enz resistance after an average of 4.8 months response to Enz. (Scher, et al. 2012 N Engl J Med 367, 1187-1197; Dhingra, et al. 2013 Mini Rev Med Chem 13, 1475-1486.)

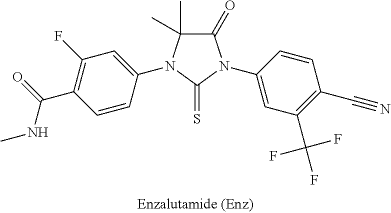 [SMILES: CNC(=O)c1ccc(N2C(=S)N(c3ccc(C#N)c(C(F)(F)F)c3)C(=O)C2(C)C)cc1F]

Since most CRPC patients received Enz may develop the Enz-resistance, and more studies indicated that it might involve multiple mechanism(s) to develop such resistance. For example, Enz or its 2^{nd} generation ARN509 might induce an AR point mutation at AR876 (a missense mutation of phenylalanine 876 to leucine in the Ligand-Binding-Domain (LBD) of AR, named AR-F876L) that no longer sensitive to Enz treatment. (Joseph et al. 2013 Cancer Discov 3, 1020-1029; Korpal et al. 2013 Cancer Discov 3, 1030-1043.) Early clinical data revealed that 3 of 29 CRPC patients received ARN509 treatment had the AR-F876L mutant. Balbas et al. confirmed these findings by showing AR-F876L could convert Enz into an AR agonist and ectopic expression of AR-F876L reversed the growth inhibition of Enz treatment. (Balbas et al. 2013 eLife 2, e00499.)

Enz might also increase the GR signals in a subset of PCa cells due to relief of AR-mediated feedback repression of GR expression. GR and AR are closely related members of the nuclear receptor superfamily with similar DNA-binding-domain, and it is possible that the GR could replace some AR roles during development of Enz-resistance. Importantly, Arora et al found the GR agonist dexamethasone was sufficient to confer EnzR, whereas a GR antagonist could partially restore sensitivity. (Arora et al. 2013 Cell 155, 1309-1322; Yemelyanov et al. 2012 Cell Cycle 11, 395-406; Sharifi 2014 N Engl J Med 370, 970-971.)

However, the development of AR splicing variant ARv7 may represent the key factor for the development of Enz-resistance as recent clinical studies from CRPC patients demonstrated that 39% of metastatic CRPC patients treated with Enz had detectable ARv7 in their circulating tumor cells (CTCs), and these ARv7-positive patients had lower PSA response rates than ARv7-negative patients with shorter PSA progression-free survival (median, 1.4 months vs. 6.0 months), suggesting CRPC patients with ARv7 might have poor response to Enz treatment, and Enz treatment might enhance ARv7 expression. (Antonarakis et al. 2014 N Engl J Med 371, 1028-1038.)

ARv7 is constitutively active and reported to regulate a transcriptional program that is similar but not identical to that of AR in CRPC, and adding Enz could increase the expression of constitutively active ARv7 that might transactivate AR target genes to promote CRPC progression in the castration level androgen. (Hu et al. 2009 Cancer research 69, 16-22; Guo et al. 2009 Cancer research 69, 2305-2313; Hu et al. 2012 Cancer research 72, 3457-3462; Yamashita et al. 2012 Neoplasia 14, 74-83; Sun et al. 2010 The Journal of clinical investigation 120, 2715-2730; Lai et al. 2013 The American journal of pathology 182, 460-473.)

At least 2 newly developed compounds have been demonstrated to be able to target the ARv7. The first one is the AR degradation enhancer ASC-J9®, that could selectively degrade AR protein in some, but not all cell types, with little side effects in all in vivo mice studies. Importantly, ASC-J9®, and not the anti-androgens Enz or Casodex, could degrade both wild type AR and the AR variant ARv7 or AR mutants including AR-F876L. (Yamashita et al. 2012 Neoplasia 14, 74-83; Lai et al. 2013 The American journal of pathology 182, 460-473; Miyamoto et al. 2007 J Natl Cancer Inst 99, 558-568; Lin et al. 2013 J Biol Chem 288, 19359-19369; Lin et al. 2013 Cell Death Dis 4, e764; Yang et al. 2007 Nat Med 13, 348-353; Wu et al. 2010 Sci Transl Med 2, 32ra35; He et al. 2014 Cancer research 74, 4420-4430; Izumi et al. 2013 EMBO Mol Med 5, 1383-1401.)

Niclosamide, an anti-helminthic compound, was the 2^{nd} compound that identified as a ARv7 inhibitor to suppress the PCa progression. Liu et al found niclosamide could suppress ARv7 protein expression via a proteasome-dependent pathway to suppress the PCa cell growth in vitro and in vivo. (Liu et al. 2014 Clinical cancer research: an official journal of the American Association for Cancer Research 20, 3198-3210.)

MicroRNA (miR)-124 has recently identified as a tumor suppressor to suppress the PCa progression, and miR-124 could also down-regulating ARv7 along with EZH2 and Src signals.

While all above-mentioned compounds may have capacity to target the ARv7 in various in vitro PCa cell lines and in vivo mouse models, none of these compounds are ready to be used in CRPC patients that already developed Enz resistance. In contrast, Cis has been widely used as chemotherapy in various tumors, including PCa, since approved in 1978 by the U.S. Food and Drug Administration. (Huan, et al. 1999 Am J Clin Oncol 22, 471-474; Kaku et al. 2006 Acta Med Okayama 60, 43-49.) The current used dose of Cis in chemotherapy by the medical oncologists to treat various solid tumors ranges from 70 mg/m² every 3 weeks to 20 mg/m² daily×5 days every 3 weeks. Radiation oncologists also used 20 mg/m² weekly (equal to 6.75 mg/kg mouse) as a radiation-sensitizing agent. (Reed, et al. 1988 Carcinogenesis 9, 1909-1911; Motzer et al. 1994 Cancer 73, 2843-2852; Homma et al. 2011 Jpn J Clin Oncol 41, 980-986.)

Compared to the sub-dose used in the in vitro cell lines and in vivo mouse model (3.5 mg/kg mouse body weight), the potential unwanted side effects of using these sub-dose of Cis to treat EenR tumors shall be minimal.

Interestingly, all previous Cis studies for its effect to suppress tumor progression all focused on its capacity to bind/crosslink to DNA to trigger the cell apoptosis that might involving the altering the DNA repair/damage system. Its linkage to alter the AR signals, especially the degradation of AR splicing mutant ARv7, however, remains unknown. The results showing Cis may increase/restore Enz sensitivity to further suppress EnzR cell growth and may delay the development of Enz-resistance in CRPC may represent a new and novel finding that can extend the Cis clinical application to those CRPC patients received Enz treatment.

The potential side effects of Cis used in current chemotherapy include myelosuppression, asthenia and gastrointestinal disorder, as well as long-term cardiac, renal and neurological consequences, which may result in its discontinuation and limit therapeutic efficacy. (Perez 1998 Eur J Cancer 34, 1535-1542; Rossi, et al. 2005 Expert Opin Drug Saf 4, 1051-1067.)

However, as disclosed herein, one may be able to extend patients survival rate and reduce those unwanted side effects by combining Enz with relative low dose of Cis to treat those CRPC patients that already developed EnzR. Alternatively, one may be able to use Carboplatin to replace Cis since Carboplatin may share similar anti-tumor effects with Cis yet it may have less side effect than Cis. (Hager, et al. 2016 Ann Oncol 27, 975-984.) Importantly, Carboplatin like Cis, can also target ARv7 (and AR) to increase Enz-sensitivity to further suppress the EnzR cell proliferation.

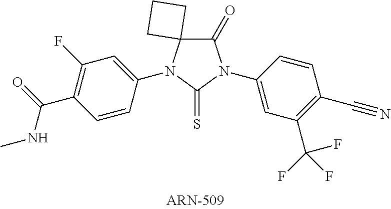 [SMILES: CNC(=O)c1ccc(N2C(=S)N(c3ccc(C#N)c(C(F)(F)F)c3)C(=O)C23CCC3)cc1F]

It has now been unexpectedly discovered, as disclosed herein, that Cis, a chemotherapy agent widely used to suppress tumor progression due to its capacity to bind to and cause crosslinking of DNA, can be used to prevent, delay, reduce and/or reverse EnzR resistance and to increase, restore and/or prolong the effective treatment of prostate cancer with anti-androgen compounds, such as Enzalutamide and its derivative ARN-509.

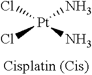

Thus, the present invention enables a novel and ready therapy to suppress the Enz-resistant CRPC progression and to extend survival of CRPC patients. In certain embodiments of the invention, as discussed in details herein, a combination of Cis and Enz may be used at the beginning of therapy. In certain other embodiments, sequential treatment with Enz therapy at the beginning may be followed by addition of Cis at or after confirmation of EnzR (e.g., when decreased PSA start to rise again).

In one aspect, the invention generally relates to a pharmaceutical composition, which include: a first compound of Formula (I)

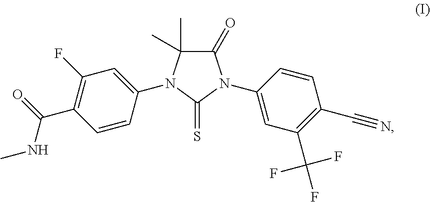 [SMILES: CNC(=O)c1ccc(N2C(=S)N(c3ccc(C#N)c(C(F)(F)F)c3)C(=O)C2(C)C)cc1F]

or a pharmaceutically acceptable salt, ester or pro-drug thereof, and a second compound of Formula (II)

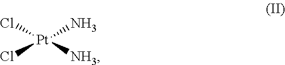

or a pro-drug thereof, wherein each of the first and second compounds is present in an amount such that the pharmaceutical composition is effective in the treatment of prostate cancer, or a related disease or condition thereof, in a mammal, including a human, and a pharmaceutically acceptable carrier.

In certain embodiments of the pharmaceutical composition, each of the first and second compounds is present in an amount such that the pharmaceutical composition is effective in preventing the onset of or delaying the development of drug resistance.

In certain embodiments, each of the first and second compounds is present in an amount such that the pharmaceutical composition is effective in reducing or reversing drug resistance.

Any suitable ratios of the first compound to the second compound may be employed as determined by the specification indication and patient profile, for example, a molar ratio of the first compound to the second compound is from about 10:1 to about 1:10 (e.g., about 8:1 to about 1:8, about 7:1 to about 1:7, about 6:1 to about 1:6, about 5:1 to about 1:5, about 4:1 to about 1:4, about 3:1 to about 1:3, about 2:1 to about 1:2, about 1.5:1 to about 1:1.5, about 1:1.)

Dosages of Enz may be any suitable amount as determined by the medical professional, for example, about 80 mg to about 120 mg daily by oral administration. When used in combination with Cis, including co-administration of separate unit dosage forms or in a single dosage form having both drug, the amount of Enz may be adjusted as needed. By way of example, in the case of 100 mg of Enz daily, a 1:1 molar ratio of Enz (m.w. 464.44 g/mol):Cis (m.w. 300.01 g/mol) corresponds to a daily dose of approximately 65 mg of Cis. Again by way of example, in the case of 100 mg of Enz daily, a 3:1 molar ratio of Enz:Cis corresponds to a daily dose of approximately 22 mg of Cis. Again by way of example, in the case of 100 mg of Enz daily, a 5:1 molar ratio of Enz:Cis corresponds to a daily dose of approximately 13 mg of Cis.

In certain embodiments, the pharmaceutical composition is suitable for oral administration.

In certain embodiments, the pharmaceutical composition is suitable for intravenous, intramuscular, or subcutaneous administration.

In another aspect, the invention generally relates to a unit dosage form comprising the pharmaceutical composition disclosed herein.

In certain embodiments, the unit dosage form is in the form of a tablet or capsule suitable for oral administration.

In certain embodiments, the unit dosage form is in the form of a liquid solution or suspension suitable for intravenous, intramuscular, or subcutaneous administration.

In yet another aspect, the invention generally relates to a method for treating prostate cancer or a related disease or condition thereof. The method includes administering to a subject in need thereof the pharmaceutical composition disclosed herein.

In certain embodiments of the method, the pharmaceutical composition is administered in combination with one or more other anti-cancer agents.

In yet another aspect, the invention generally relates to a method for treating prostate cancer or a related disease or condition thereof. The method includes administering to a subject in need thereof a first compound of Formula (I):

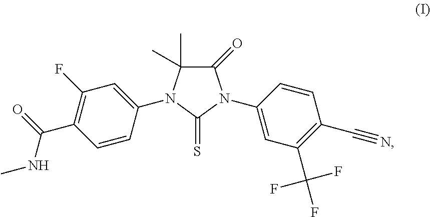 [SMILES: CNC(=O)c1ccc(N2C(=S)N(c3ccc(C#N)c(C(F)(F)F)c3)C(=O)C2(C)C)cc1F]

or a pharmaceutically acceptable salt, ester or pro-drug thereof, and a second compound of Formula (II)

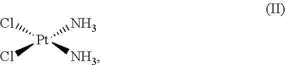

or a pro-drug thereof, in amounts effective in the treatment of prostate cancer or a related disease or condition thereof in a mammal, including a human.

In certain embodiments of the method, the subject has been detected of the development of drug resistance to the first compound.

In certain embodiments of the method, the subject has not been detected of the development of drug resistance to the first compound.

In certain embodiments of the method, the second compound is administered simultaneously with the first compound, or subsequently after the first the administration of the first compound.

In certain embodiments of the method, the first and/or the second compound is administered orally.

In certain embodiments of the method, the first and/or the second compound is administered intravenously, intramuscularly, or subcutaneously.

In certain embodiments, the drug resistance is related to Arv7 and/or AR mutant.

In yet another aspect, the invention generally relates to a method for treating prostate cancer or a related disease or condition thereof. The method includes: administering to a subject in need thereof a first compound of Formula (I)

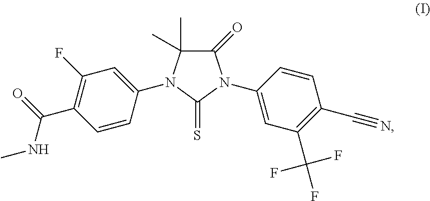 [SMILES: CNC(=O)c1ccc(N2C(=S)N(c3ccc(C#N)c(C(F)(F)F)c3)C(=O)C2(C)C)cc1F]

or a pharmaceutically acceptable salt, ester or pro-drug thereof, in an amount effective in the treatment of prostate cancer or a related disease or condition thereof in a mammal, including a human; monitoring the subject to detect a development of drug resistance to the first compound; upon the subject being detected of the development of drug resistance to the first compound, administering to the subject a second compound of Formula (II)

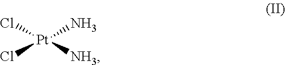

or a pro-drug thereof, in an amount effect to reduce or eliminate drug resistance to the first compound; and monitoring the subject to detect a level of drug resistance to the first compound.

In certain embodiments, the method further includes: upon the subject being detected of the development of drug resistance to the first compound, continuing to administer the subject the first compound.

In certain embodiments, the method further includes: upon the subject being detected of the development of drug resistance to the first compound, halting the administration of the first compound, and upon the subject being detected of a substantial reduction or disappearance of drug resistance, re-starting the administration of the first compound.

In certain embodiments, the first and/or the second compound are administered orally.

In certain embodiments, the first and/or the second compound are administered intravenously, intramuscularly, or subcutaneously.

In certain embodiments, the drug resistance is related to Arv7 and/or AR mutant. In certain embodiments, detecting a development of drug resistance to the first compound is by measuring ARv7.

In yet another aspect, the invention generally relates to a method for treating a drug resistance in connection with a cancer treatment. The method includes administering to a subject in need thereof a compound of Formula (II)

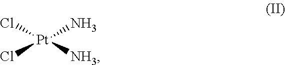

or a pro-drug thereof, in an amount effective to prevent, delay, reduce or reverse resistance to a treatment of prostate cancer, or a related disease or condition thereof in a mammal, including a human.

In certain embodiments, the method is effective in preventing the onset of or delaying the development of drug resistance.

In certain embodiments, the method is effective in reducing or reversing drug resistance.

In certain embodiments of the method, the drug resistance is related to Arv7 and/or AR mutant.

In certain embodiments, the method is the resistance is to a compound having Formula (I)

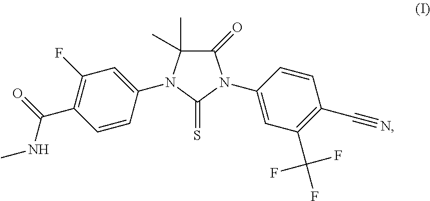 [SMILES: CNC(=O)c1ccc(N2C(=S)N(c3ccc(C#N)c(C(F)(F)F)c3)C(=O)C2(C)C)cc1F]

or a pharmaceutically acceptable salt, ester or pro-drug thereof.

In certain embodiments of the method, the pharmaceutical composition of the invention is administered in combination with one or more other anti-cancer agents. In certain embodiments, the one or more other anti-cancer agents is a chemotherapeutic agent. Examples of chemotherapeutic agents include Erlotinib (TARCEVA®, Genentech/OSI Pharm.), Bortezomib (VELCADE®, Millennium Pharm.), Fulvestrant (FASLODEX®, AstraZeneca), Sutent (SU11248, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (TYKERB®, GSK572016, Glaxo Smith Kline), Lonafarnib (SCH 66336), Sorafenib (BAY43-9006, Bayer Labs), and Gefitinib (IRESSA®, AstraZeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (Angew Chem. Intl. Ed. Engl. (1994) 33: 183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esonibicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamniprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (doxetaxel; Rhone-Poulenc Rorer, Antony, France); chloranmbucil; GEMZAR® (gemcitabine); 6-thioguanine; mercaptopurine; methotrexate; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylomithine (DMFO); retinoids such as retinoic acid; and pharmaceutically acceptable salts, acids and derivatives of any of the above.

The following examples are meant to be illustrative of the practice of the invention, and not limiting in any way.

### Examples

### Cisplatin Decreases the Expression of ARv7, AR876 and AR in Multiple Enz-Sensitive (Enz-S) and Enz-Resistant (Enz-R) CRPC Cells

After PCa patients developed CRPC, the current standard therapy to further suppress the CRPC may involve the ADT with newly developed Enz to prevent androgens from binding to the AR or using ABI to further suppress the androgen bio-synthesis in renal. However, most patients may still develop the Enz-resistance after extend extra 4.8-months survival. Results from in vitro cell lines and human clinical surveys suggested that the induction of Enz-resistance may involve the increase of the AR splicing mutant ARv7 that can transactivate AR in the absence of androgens, and targeting this Enz-induced ARv7 may help us to overcome/decrease the Enz-resistance to further improve CRPC survival rate.

First produced were the EnzR CRPC cells via chronic culture of CRPC C4-2 cells in media containing increasing Enz concentrations from 10 to 30 nM for 1 year (named as EnzR1-C4-2). Then, this EnzR1-C4-2 cell line was used with their parental C4-2 cells that are still sensitive to Enz (named as EnzS1-C4-2) to screen FDA-approved drugs for their capacity to target the ARv7 expression.

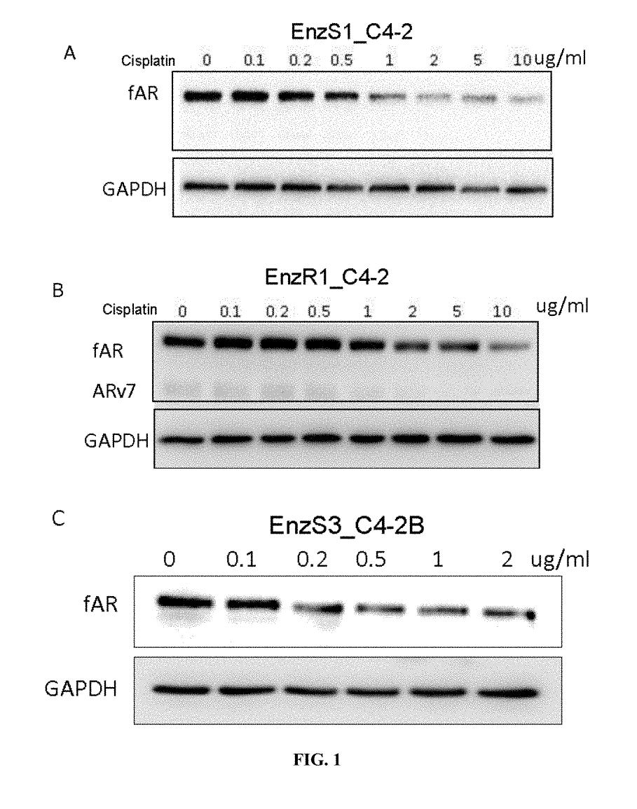

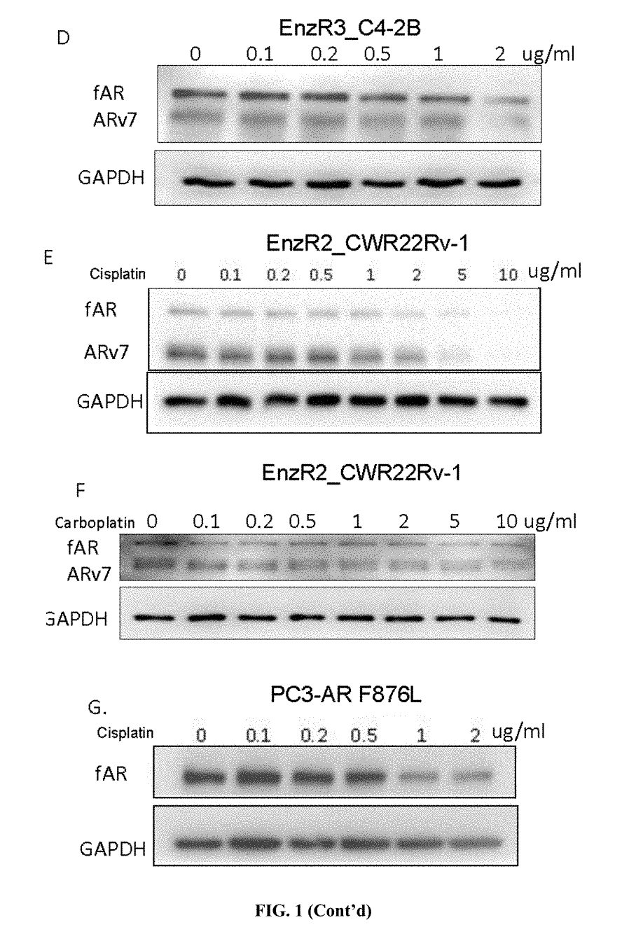

Interestingly and unexpectedly, it was found that the Cisplatin (Cis), a current available drug used in chemotherapy to induce several tumor cell apoptosis via crosslinking DNA to alter the DNA repair-damage signals, could suppress the AR in both EnzS1-C4-2 (FIG. 1A, ARv7 was not detected since little ARv7 is expressed in EnzS1-C4-2 cells) and EnzR1-C4-2 cells (FIG. 1B, the diminishing ARv7 was detected upon increasing Cis in EnzR1-C4-2 cells). Similar results were also obtained when the pair of EnzS1-C4-2 and EnzR1-C4-2 cells were replaced with EnzS3-C4-2B/EnzR3-C4-2B cells (FIG. 1C-D), as well as in the EnzR2-CWR22Rv1 cells which expressed both AR and ARv7 (FIG. 1E). Importantly, it was found Carboplatin, a new Cis analog with less side effect that used widely in current chemotherapy, could also decrease the expression of ARv7 (and AR) in EnzR2-CWR22Rv1 cells (FIG. 1F).

Furthermore, it was found that Cis can also decrease the Enz-increased AR mutant AR876 that might also contribute to the development of Enz-resistance in PC3 cells stably transfected with AR876-cDNA (FIG. 1G). In contrast, Cis had little effect to decrease other nuclear receptors including glucocorticoid receptor (GR) and estrogen receptor b (ERb) expression.

Together, results from FIG. 1A-G indicate that Cis can target selectively the expression of ARv7, AR876 and AR in multiple Enz-R CRPC cells.

### Cisplatin Restores/Increase the Enz-Sensitivity to Further Suppress the Enz-R CRPC Cells

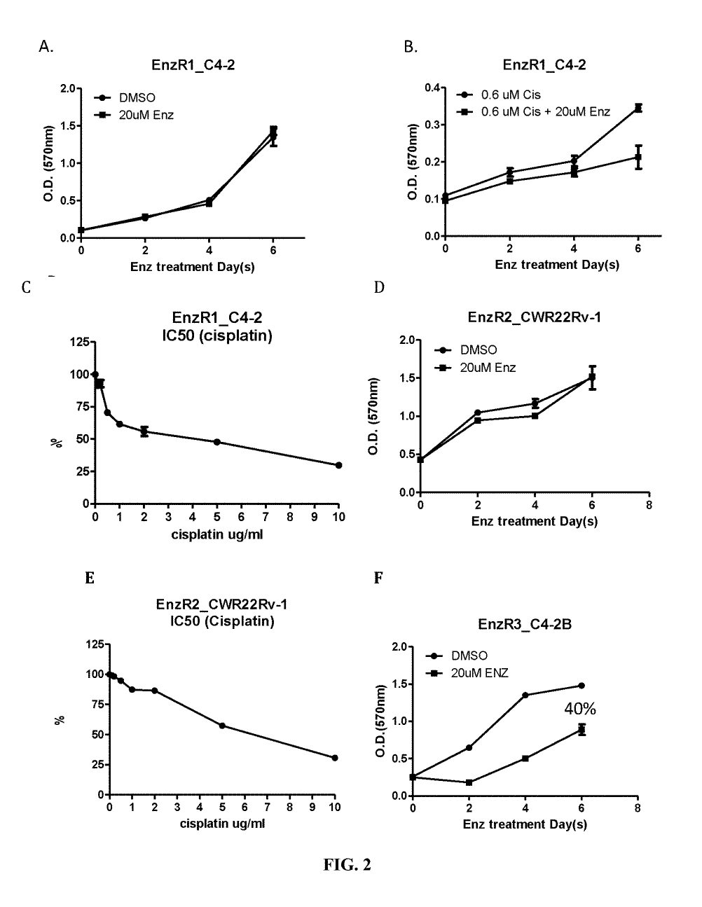

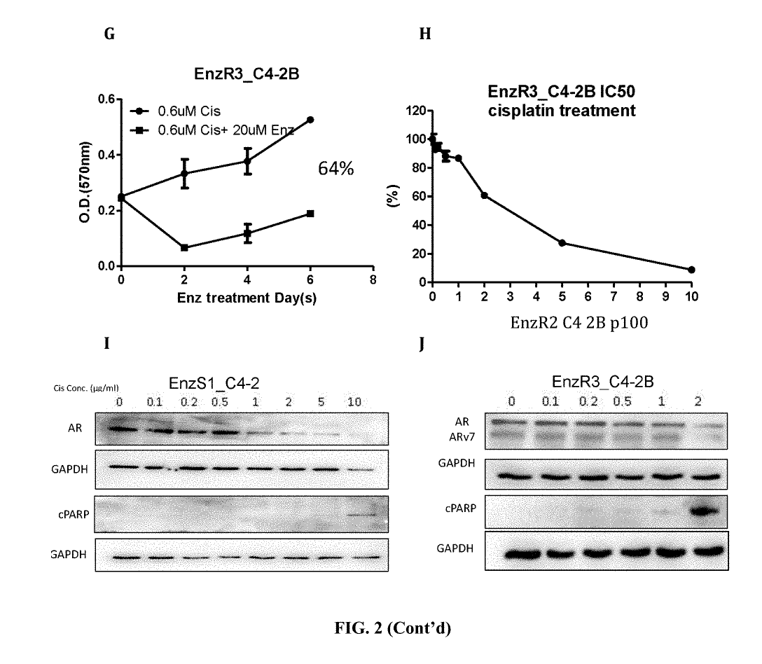

To further examine the impacts of Cis-suppressed ARv7 expression in Enz-S and Enz-R CRPC cells, then added was a different dose of Cis in the EnzR1-C4-2 cell line. The results revealed that adding 20 uM Enz alone has little effect to suppress the EnzR1-C4-2 cell growth (FIG. 2A). In contrast, combined 20 uM Enz with 2 ug/mL (6.6 uM) Cis could suppress EnzR1-C4-2 cell growth (FIG. 2B) with IC50 near 5 ug/mL (16.5 uM) (FIG. 2C).

Similar results were also obtained when EnzR1-C4-2 cells with EnzR2-CWR22Rv1 cells (FIG. 2D) were replaced with IC50 at 5 ug/mL (16.5 uM) (FIG. 2E) and EnzR3-C4-2B (FIG. 2F-G) with IC50 IC50 at 3.3 ug/mL (10.9 uM) (FIG. 2H). Importantly, combined 20 uM Enz with Carboplatin may also suppress EnzR1-C4-2 cell growth.

Next was to study if Cis can restores/increase the Enz-sensitivity to further suppress the Enz-R CRPC cells is via targeting ARv7 and not via its classic inducing the apoptosis signals. Results from EnzS1-C4-2 cells revealed that Cis at 1-2 ug/mL could degrade AR yet at this dose still failed to alter the cell apoptosis as indicated little change in cPARP (FIG. 2I). Similar results were also observed in EnzR3-C4-2 cells showing Cis at 1 ug/mL may degrade AR yet at this dose still failed to alter the cell apoptosis as indicated little change in cPARP (FIG. 2J). Additionally, adding ARv7 can reverse/block the Cis suppression cell growth in EnzR1-C4-2 cells and EnzR3-C4-2B cells.

Together, results from 3 different Enz-R CRPC cells (FIG. 2A-J) all suggest that adding Cis can function via targeting the ARv7 to restore/increase the Enz-sensitivity to further suppress the Enz-R CRPC cells

### Cisplatin Also Delays the Development of Enz-Resistance in CRPC Cells Receiving Enz

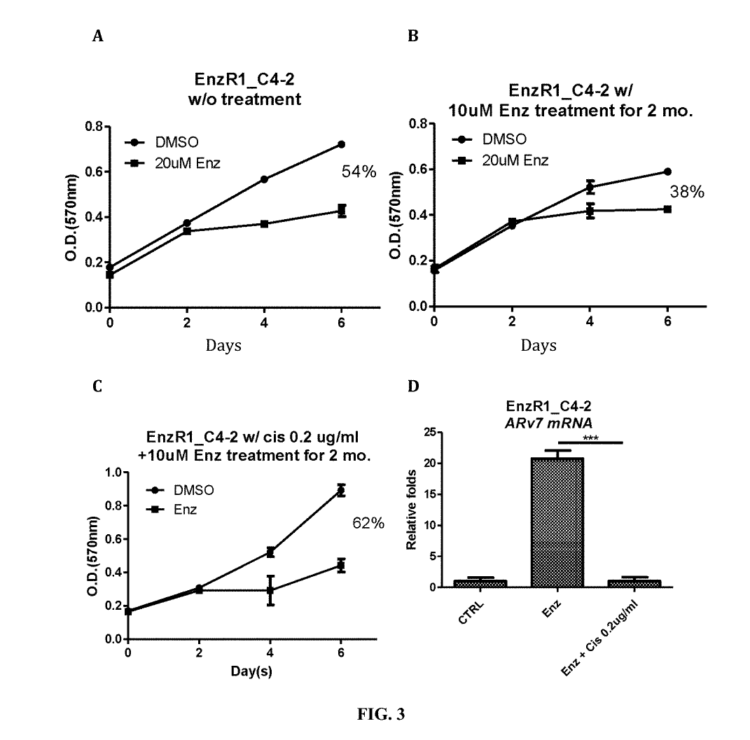

Next examined was whether Cis may also delay the development of Enz-resistance in CRPC cells receiving Enz. Enz-S1-C4-2 was first treated with 10 uM Enz alone vs 10 uM Enz plus 0.2 ug/mL (0.7 uM) Cis for 2 months, and then compared their Enz-sensitivity. As shown in FIG. 3A, adding 10 uM Enz alone in Enz-S1-C4-2 cells decreased the Enz sensitivity from 54% to 38% (FIG. 3A vs 3B). In contrast, adding 10 uM Enz plus 0.2 ug/mL (0.66 uM) delayed/reversed the Enz-sensitivity from 54% to 62% (FIG. 3A vs 3C). Further compared was the ARv7 expression in these two cells, and results revealed that adding Enz led to increase ARv7 mRNA expression and adding Cis (0.2 ug/mL) suppressed the Enz-increased ARv7 significantly (FIG. 3D).

Together, results from FIG. 3A-D indicate that Cis can also delay the development of Enz-resistance in CRPC cells receiving Enz.

### Mechanism Dissection why Cis can Decrease the ARv7 Protein Expression: Via Altering the Ubiquitination to Affect the ARv7 Protein Stability

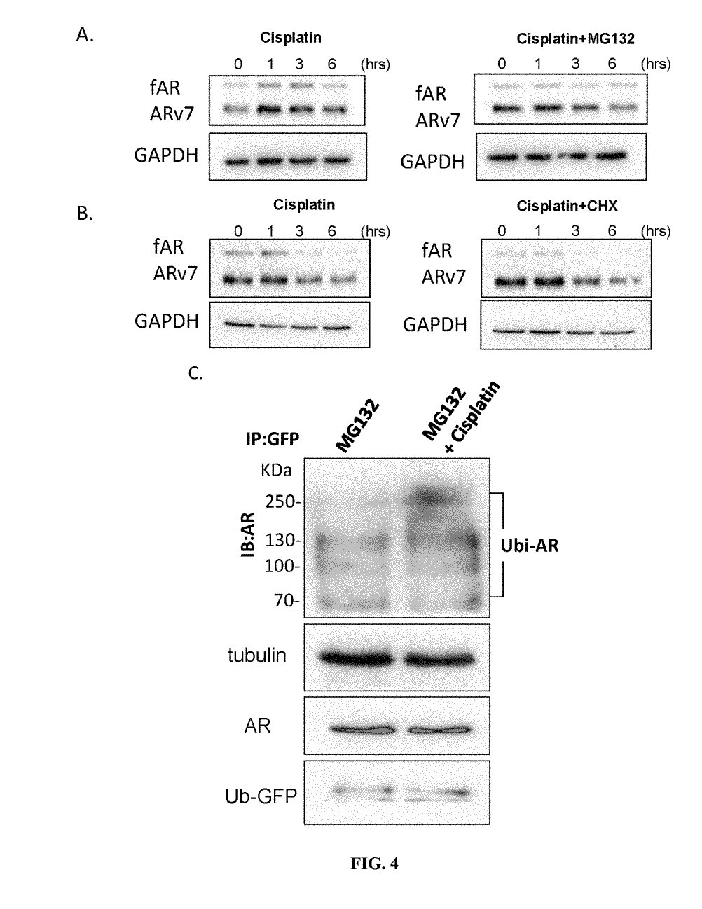

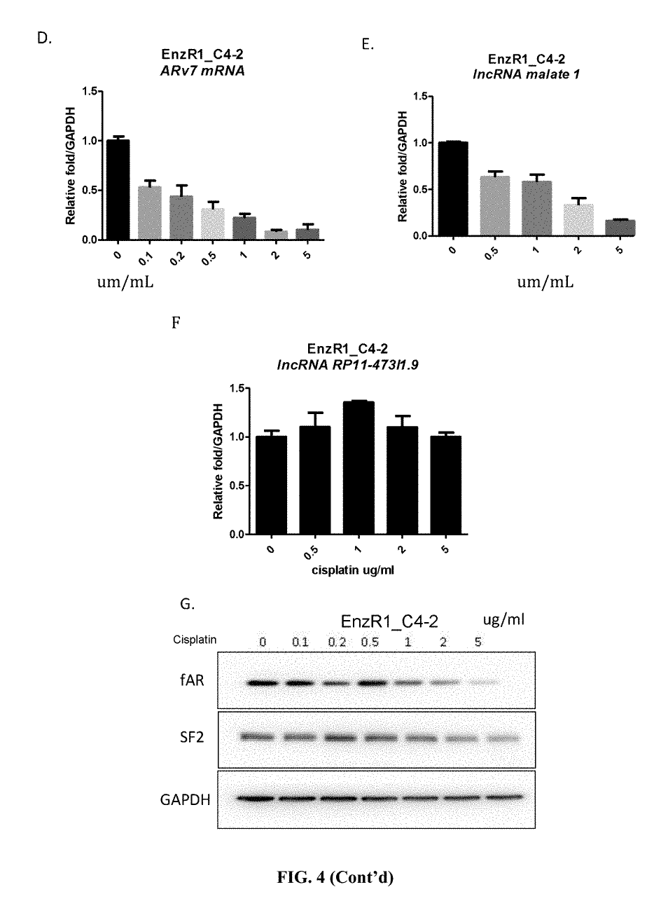

To further dissect the mechanism(s) why Cis can decrease ARv7 (and AR) protein expression, first assayed was the Cis effects on ARv7 (and AR) protein stability with protein biosynthesis inhibitor, cycloheximide (CHX) and proteasome inhibitor MG132 in EnzR2-CWR22Rv1 cells. The results revealed that Cis could degrade ARv7 (and AR) after 6 hrs treatment (FIG. 4A), and adding MG132 with Cis 6 hrs led to attenuate/inhibit the effect of Cis on ARv7 (and AR) degradation (FIG. 4B). It was also found adding Cis plus CHX can facilitate ARv7 (and AR877) degradation compared to CHX alone in CWR22Rv1 cells (FIG. 4C).

Then examined was whether ubiquitination involves in ARv7 (and AR) degradation. First added were AR and ubiquitin-GPF in 293T cells, and then applied the immunoprecipitation assay to examine Cis effect on AR-ubiquitination. The results revealed that Cis may increase the AR-ubiquitin complex (FIG. 4D).

Together, results from FIG. 4A-D suggest that Cis may degrade ARv7 (and AR) through increasing ubiquitination of AR.

Mechanism Dissection why Cis can Decrease the ARv7 Protein Expression: Via Altering the MALAT1/SF2 RNA Splicing Complex to Decrease the ARv7 mRNA Expression

Since recent studies indicated that Enz might function through inducing LncRNA-Malat1 and its associated SF2 RNA splicing protein (named as Malat1/SF2 RNA splicing complex) to increase ARv7 biosynthesis/expression, it was interesting to see if Cis may also function through altering this Malat1/SF2 RNA splicing complex to decrease the ARv7 mRNA biosynthesis/expression. As expected, it was found that ARv7 and Malat1 mRNA expression were decreased in dose-dependent manner after Cis treatment in EnzR1-C4-2 cells (FIG. 4E-F). In contrast, it was found Cis had little effect on other lncRNA RP11-473I1.9 expression (FIG. 4G). Also examined was the SF2 at protein level after Cis treatment. Cis may decrease SF2 protein expression in a dose-dependent manner.

Importantly, using interruption approach via overexpressing Malat1 can reverse/block the Cis-induced ARv7 expression in EnzR1-C4-2 cells, indicating Malat1 indeed plays key roles to mediate Cis-induced ARv7 biosynthesis/expression.

Together, results from FIG. 4E-G suggest that in addition to increase the ubiquitination of AR, Cis can also function through altering the Malat1/SF2 RNA splicing complex to decrease the ARv7 mRNA biosynthesis/expression.

Mechanism Dissection why Cis can Decrease the ARv7 Protein Expression: Via Decreasing the AR mRNA Expression at Transcriptional Level

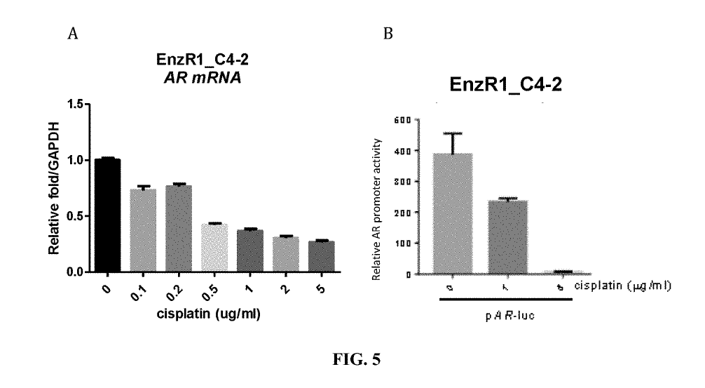

In addition to function via altering the AR-ubiquitination to decrease ARv7 (and AR) protein expression and modulating the Malat1/SF2 RNA splicing complex to decrease ARv7 mRNA biosynthesis/expression, it is interesting to see the potential 3^{rd} molecular mechanism at transcriptional regulation to alter the AR expression for Cis to decrease ARv7 mRNA expression. It was first found that Cis could also decrease AR mRNA in a dose-dependent manner (FIG. 5A). A 3.6 kb AR promoter was then constructed into pGL3 luciferase vector (pAR-luc) and transfected pAR-luc into EnzR2-CWR22Rv1 cells for luciferase assay. The results revealed that 1 ug/mL Cis can also decrease AR mRNA expression at transcriptional level via altering AR promoter activity (FIG. 5B).

### New Therapy to Further Suppress EnzR Progression in In Vivo Mouse Model

To prove the above in vitro results from multiple cell lines in the in vivo mouse model, the EnzR2-CWR22Rv1 cells were orthotopically xenografted into nude mice. After tumor reaches≧200 m³, mice were then castrated and injected Enz (3.5 mg/kg/every other day for i.p.) with or without adding Cis (3.5 mg/kg/weekly for i.v.) for 3 weeks. Adding Cis can increase/restore the Enz sensitivity to further suppress the EnzR3-C4-2B tumor progression. Results from Immunohistochemistry staining also revealed the EnzR2-C4-2B tumor received Cis+Enz had lower expression of ARv7 (and AR), as well as Malat1 and SF2.

Together, results from in vivo mouse model confirm the in vitro cell lines data results showing Cis can increase the Enz-sensitivity to further suppress the EnzR CRPC cell growth.

In conclusion, the results in in vitro multiple EnzR-CRPC cells and in vivo mouse models not only unveil Cis unrecognized mechanism to degrade ARv7 (and AR) via targeting the Malat1/SF2 and ubiquitination signals, it may also provide a novel and ready available therapy to suppress the EnzR-CRPC progression to further extend CRPC patients survival.

### EXPERIMENTAL

### Generation of Acquired EnzR CRPC Cell Models

Prostate cancer cell lines, C4-2 and CWR22Rv1, were obtained from American Type Culture Collection (ATCC) and maintained in RPMI 1640 media (GIBCO) supplemented with 10% Fetal Bovine Serum (FBS). Enz-resistant clones, EnzR C4-2H, and Enz C4-2J were selected by culturing cells with Enz in a dose-escalation. Initial culture was at 10 μM Enz. After sensitive clones were no longer present and surviving cells repopulated the dish, the concentration of Enz was increased gradually. EnzR C4-2 cells were further exposed to 20 μM. Cell proliferation rates were analyzed by MTT assay monthly after Enz treatment. The process of acquired drug resistance took 12 months.

### MTT Cell Proliferation Assay

Cells were seeded in 24-well plates (5×10³ cells/500 μL media per well) and cultured for 0, 2, 4 and 6 days. Cells were harvested and cell numbers and absorbance were calculated and recorded after using yellow tetrazolium MTT (3-(4, 5-dimethylthiazolyl-2)-2,5-diphenyltetrazolium bromide agent.

### AR Degradation and Ubiquitination

For protein degradation, EnzR C4-2 cells were treated with 1 μg/mL Cisplatin for 6 hr. Cycloheximide or MG132 was added for another 4 hrs. For ubiquitination, human kidney epithelial HEK293T cells were grown in DMEM media with 10% FCS. Cells were plated at ˜60% confluency and pGFP-ubiquitin, pAR were transiently transfected into cells using CaCl₂ reagent. After 48 hrs. of transfection, cells were treated with 1 μg/mL Cisplatin for 6 hrs. Protease inhibitor, MG132, was added for another 4 hrs. Cell extracts were analyzed for AR degradation or AR-ubiquitination by using western blot.

### RNA Extraction and Quantitative Real-Time PCR Analysis

Total RNAs were isolated using Trizol reagent (Invitrogen, Grand Island, N.Y.). 2 μg of total RNA was subjected to reverse transcription using Superscript III transcriptase (Invitrogen). Quantitative real-time PCR (qRT-PCR) was conducted using a Bio-Rad CFX96 system with SYBR green to determine the mRNA expression level of a gene of interest. Expression levels were normalized to the expression of GAPDH.

### Western Blotting

Cells were lysed in lysis buffer and proteins (50 μg) were separated on 10% SDS/PAGE gel and then transferred onto PVDF membranes (Millipore, Billerica, Mass.). After blocking membranes with 5% milk, they were incubated with appropriate dilutions of specific primary antibodies (anti-AR (N-20), anti-SF2, anti-GFP, anti-GAPDH, anti-a-tubulin). The blots were incubated with HRP-conjugated secondary antibodies and visualized using ECL system (Thermo Fisher Scientific, Rochester, N.Y.).

### Prostate Orthotopic Implantation

1×10⁶ parental C4-2 or EnzR C4-2 cells were directly injected with matrigel (1:1) into the anterior prostates of nude mice. After anesthesia, the skin was lifted with a pair of blunt forceps and cut along the midline with a pair of scissors creating an incision approximately a half-inch in length. Location of the anterior prostates was identified by viewing it with an IVIS system.

### Statistics

All statistical analyses were carried out with SPSS 16.0 (SPSS Inc., Chicago, Ill.). The data values were presented as the mean±SD. Differences in mean values between two groups were analyzed by two-tailed Student's t test. p≦0.05 was considered statistically significant.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description herein, specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples disclosed herein are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. The following examples contain important additional information, exemplification and guidance that can be adapted to the practice of this invention in its various embodiments and equivalents thereof.

1. A pharmaceutical composition comprising:
a first compound of Formula (I) 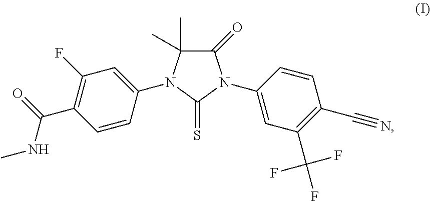 [SMILES: CNC(=O)c1ccc(N2C(=S)N(c3ccc(C#N)c(C(F)(F)F)c3)C(=O)C2(C)C)cc1F]
or a pharmaceutically acceptable salt, ester or pro-drug thereof, and
a second compound of Formula (II) 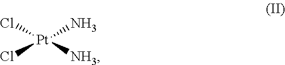
or a pro-drug thereof, wherein each of the first and second compounds is present in an amount such that the pharmaceutical composition is effective in the treatment of prostate cancer, or a related disease or condition thereof, in a mammal, including a human, and a pharmaceutically acceptable carrier.

2. The pharmaceutical composition of claim 1, wherein each of the first and second compounds is present in an amount such that the pharmaceutical composition is effective in preventing the onset of or delaying the development of drug resistance.

3. The pharmaceutical composition of claim 1, wherein each of the first and second compounds is present in an amount such that the pharmaceutical composition is effective in reducing or reversing drug resistance.

4. The pharmaceutical composition of claim 1, wherein the molar ratio of the first compound to the second compound is from about 10:1 to about 1:10.

5. The pharmaceutical composition of claim 1, wherein the molar ratio of the first compound to the second compound is from about 5:1 to about 1:5.

6. The pharmaceutical composition of claim 1, being suitable for oral administration.

7. The pharmaceutical composition of claim 1, being suitable for one or more of intravenous, intramuscular, or subcutaneous administration.

8. A unit dosage form comprising the pharmaceutical composition of claim 1.

9. The unit dosage form of claim 8, being in the form of a tablet or capsule suitable for oral administration.

10. The unit dosage form of claim 8, being in the form of a liquid solution or suspension suitable for one or more of intravenous, intramuscular, or subcutaneous administration.

11. A method for treating prostate cancer, or a related disease or condition thereof, comprising administering to a subject in need thereof the pharmaceutical composition of claim 1.

12. The method of claim 11, wherein the pharmaceutical composition is administered in combination with one or more other anti-cancer agents.

13. A method for treating prostate cancer, or a related disease or condition thereof, comprising administering to a subject in need thereof a first compound of Formula (I) 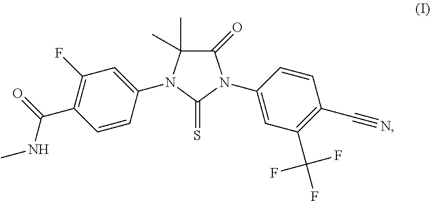 [SMILES: CNC(=O)c1ccc(N2C(=S)N(c3ccc(C#N)c(C(F)(F)F)c3)C(=O)C2(C)C)cc1F]
or a pharmaceutically acceptable salt, ester or pro-drug thereof, and a second compound of Formula (II) 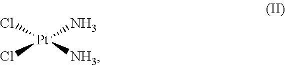
or a pro-drug thereof, in amounts effective in the treatment of prostate cancer, or a related disease or condition thereof, in a mammal, including a human.

14. The method of claim 13, wherein the second compound is administered simultaneously with the first compound, or subsequently after the first the administration of the first compound.

15. The method of claim 13, wherein the subject has been detected of the development of drug resistance to the first compound.

16. The method of claim 13, wherein the subject has not been detected of the development of drug resistance to the first compound.

17. The method of claim 15, wherein the drug resistance is related to Arv7 and/or AR mutant.

18. The method of claim 13, wherein the first and/or the second compound is administered orally.

19. The method of claim 13, wherein the first and/or the second compound is administered intravenously, intramuscularly, or subcutaneously.

20. A method for treating prostate cancer, or a related disease or condition thereof, comprising:
administering to a subject in need thereof a first compound of Formula (I) 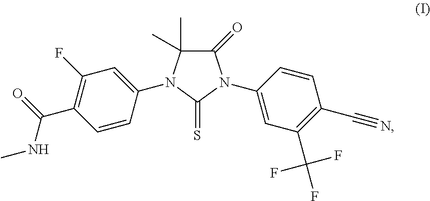 [SMILES: CNC(=O)c1ccc(N2C(=S)N(c3ccc(C#N)c(C(F)(F)F)c3)C(=O)C2(C)C)cc1F]
or a pharmaceutically acceptable salt, ester or pro-drug thereof, in an amount effective in the treatment of prostate cancer or a related disease or condition thereof in a mammal, including a human;
monitoring the subject to detect a development of drug resistance to the first compound;
upon the subject being detected of the development of drug resistance to the first compound, administering to the subject a second compound of Formula (II) 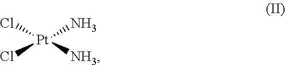
or a pro-drug thereof, in an amount effect to reduce or eliminate drug resistance to the first compound; and
monitoring the subject to detect a level of drug resistance to the first compound.

21. The method of claim 20, further comprising:
upon the subject being detected of the development of drug resistance to the first compound, continuing to administer the subject the first compound.

22. The method of claim 20, further comprising:
upon the subject being detected of the development of drug resistance to the first compound, halting the administration of the first compound, and
upon the subject being detected of a substantial reduction or disappearance of drug resistance, re-starting the administration of the first compound.

23. The method of claim 20, wherein the drug resistance is related to Arv7 and/or AR mutant.

24. The method of claim 20, wherein the first and/or the second compound is administered orally.

25. The method of claim 20, wherein the first and/or the second compound is administered intravenously, intramuscularly, or subcutaneously.

26. The method of claim 20, wherein detecting a development of drug resistance to the first compound is by measuring ARv7.

27. A method for treating a drug resistance in connection with a cancer treatment, comprising administering to a subject in need thereof a compound of Formula (II) 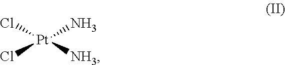
or a pro-drug thereof, in an amount effective to prevent, delay, reduce or reverse resistance to a treatment of prostate cancer, or a related disease or condition thereof in a mammal, including a human.

28. The method of claim 27, effective in preventing the onset of or delaying the development of drug resistance.

29. The method of claim 27, effective in reducing or reversing drug resistance.

30. The method of claim 27, wherein the drug resistance is related to Arv7 and/or AR mutant.

31. The method of claim 27, wherein the resistance is to a compound having Formula (I) 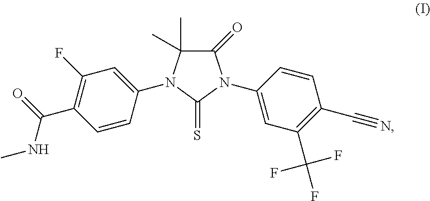 [SMILES: CNC(=O)c1ccc(N2C(=S)N(c3ccc(C#N)c(C(F)(F)F)c3)C(=O)C2(C)C)cc1F]
or a pharmaceutically acceptable salt, ester or pro-drug thereof.
